(19) United States
(12) Patent Application Publication
(10) Pub. No.: US 2018/0218780 A1
(43) Pub. Date: Aug. 2, 2018

(54) INFORMATION MANAGEMENT DEVICE, INFORMATION MANAGEMENT SYSTEM, INFORMATION MANAGEMENT METHOD, AND COMPUTER PROGRAM

(71) Applicant: NATIONAL UNIVERSITY CORPORATION NAGOYA UNIVERSITY, Nagoya-shi, Aichi (JP)

(72) Inventors: MASAAKI MIZUNO, NAGOYA-SHI (JP); AKITAKA SUGISHITA, NAGOYA-SHI (JP)

(21) Appl. No.: 15/748,000

(22) PCT Filed: Jul. 28, 2016
(86) PCT No.: PCT/JP2016/072135

§ 371 Date: Jan. 26, 2018

(30) Foreign Application Priority Data
Jul. 31, 2015 (JP) 2015-151681

(51) Int. Cl. G16H 10/60

(52) U.S. Cl. CPC G16H 10/60

(57) ABSTRACT

A device, system, method, and computer program is provided that suppresses the disappearance of personal information in a handover process of personal information. A control unit of an information management device performs a process of receiving approval or disapproval of each user for each information system to use personal information of the user; in response to receiving disapproval of a first user for a first information system, a process of causing personal information of the first user stored in a first information server provided in the first information system to be sent from the first information server to a storage server; and in response to receiving approval of the first user for a second information system, a process of causing the personal information of the first user stored in the storage server to be sent from the storage server to a second information server provided in a second information system.

### CROSS-REFERENCE TO RELATED APPLICATIONS

This application claims priority to PCT Application No. PCT/JP2016/072135, having a filing date of Jul. 28, 2016, based on Japanese Application No. 2015-151681, having a filing date of Jul. 31, 2015, the entire contents both of which are hereby incorporated by reference.

### FIELD OF TECHNOLOGY

The following relates to an information management device.

### BACKGROUND

A medical information system is generally operated in a medical institution. The medical information system is a system that enables each doctor or each medical staff to record and access medical information, such as physical examination information on each patient and information on medical drugs prescribed to each patient, so as to support the works of the doctor or the medical staff The medical information is stored in an information server provided in the medical information system (as described in, for example, Patent Literature 1). The medical information system may be operated by each medical institution such as hospital or pharmacy or may be operated by a plurality of medical institutions that cooperate with each other.

A patient is likely to change the medical institution where the patient receives services due to a move or the like. The medical information is of significance in accumulation. It is accordingly desirable to cause medical information of the patient stored in an information server (hereinafter referred to as “previous information server”) provided in a medical information system operated by a previous medical institution to be handed over to an information server (hereinafter referred to as “changed information server”) provided in a medical information system operated by a changed medical institution. A conventional practice obtains approval of the patient in writing, for example, from the viewpoint of personal information protection and causes the medical information of the patient stored in the previous information server to be handed over to the changed information server by cooperation of the previous medical institution with the changed medical institution.

### SUMMARY

Due to failed cooperation of the previous medical institution with the changed medical institution, however, the above prior practice is likely to fail to securely hand over the medical information of the patient from the previous information server to the changed information server. For example, in the case of withdrawal of approval of the patient in writing, with approval of the patient, the previous medical institution hands over the medical information of the patient stored in the previous information server to the changed medical institution and subsequently removes the medical information from the previous information server. In response to withdrawal of approval of the patient, the changed medical institution, on the other hand, removes the medical information handed over from the previous medical institution. This causes the occurrence of an event that the medical information stored in the previous information server is removed from both the previous information server and from the changed information server to disappear. In this case, even when the patient gives approval again in writing, the medical information of the patient has disappeared and is thus not handed over to the changed medical institution.

This problem is not limited to the medical information handled in the medical information system operated by the medical institution but may arise with regard to any personal information handled in any information system. The personal information includes every information regarding individuals, for example, welfare information indicating, for example, the use status of welfare services, healthcare information indicating, for example, the results of medical checks, and purchase information indicating, for example, purchase history in a net shop or in a real shop.

The present description discloses a technique that solves at least part of the problems described above.

The technique disclosed in the present description may be implemented, for example, by the following aspects.

(1) An information management device disclosed in the description hereof is connectable via a network with a storage server and with information servers respectively provided in a plurality of information systems and comprises: a communication unit configured to make communication with an external device; and a control unit. The control unit is configured to: perform a receiving process of receiving approval or disapproval of each user for each of the information systems to use personal information of the user via the communication unit; when receiving disapproval of a first user for a first information system that is one of the plurality of information systems, perform a first sending process of causing the personal information of the first user stored in a first information server provided in the first information system to be sent from the first information server to the storage server and to be stored into the storage server; and when receiving approval of the first user for a second information system that is one of the plurality of information systems, perform a second sending process of causing the personal information of the first user stored in the storage server to be sent from the storage server to a second information server provided in the second information system and to be stored into the second information server. In the process of handing over the personal information of the user stored in the first information server to the second information server, the information management device of this aspect causes the personal information of the user to be once stored into the storage server. This configuration suppresses the occurrence of an event that the personal information disappears in the process of handing over the personal information of the user stored in the first information server to the second information server. This accordingly enables the personal information to be securely handed over from the first information server to the second information server.

(2) In the information management device of the above aspect, the control unit may be configured to, when receiving the disapproval of the first user for the first information system, perform a first removal process of removing the personal information of the first user from the first information server. The information management device of this aspect securely removes the personal information of the user from the first information server that is the subject of the disapproval of the user. This configuration prevents the personal information from being used by the first information system without the user's approval.

(3) In the information management device of the above aspect, the control unit may be configured to, when receiving the approval of the first user for the second information system, perform a second removal process of removing the personal information of the first user from the storage server. The information management device of this aspect securely removes the personal information of the user from the storage server. This configuration minimizes the risk of leakage of the personal information.

(4) In the information management device of the above aspect, the control unit may be configured to, when receiving approval of a second user for a third information system that is one of the plurality of information systems, performing the second sending process of causing the personal information of the second user to be stored into a third information server provided in the third information system, and subsequently receiving approval of the second user for a fourth information system that is one of the plurality of information systems, perform a third sending process of causing the personal information of the second user stored in the third information server to be sent from the third information server to the storage server, to be further sent from the storage server to a fourth information server provided in the fourth information system, and to be stored into the fourth information server. The information management device of this aspect enables the personal information of one identical user to be readily stored in the respective information servers provided in the plurality of information systems.

(5) In the information management device of the above aspect, the control unit may receive the approval or the disapproval via the network in the receiving process. The information management device of this aspect simplifies and accelerates the process of receiving approval or disapproval, compared with a method of receiving approval or disapproval in writing.

(6) In the information management device of the above aspect, on satisfaction of a predetermined condition, the control unit may send location information for identifying the information server and the storage server where the personal information of a third user is stored, via the communication unit to a terminal device of the third user that is the external device. The information management device of this aspect enables the user to check the location of the own personal information by referring to the location information and recognize which of the information systems uses the own personal information.

(7) In the information management device of the above aspect, the control unit may be configured to perform a request receiving process of receiving a sending request of the location information from the terminal device of the third user via the communication unit. The predetermined condition may be that the sending request of the location information is received. The information management device of this aspect enables the user to obtain the location information at a desired timing.

(8) In the information management device of the above aspect, the control unit may be configured to, when receiving the disapproval of the first user for the first information system, perform a determination process of determining whether type of the personal information of the first user is a first type or a second type; and when determining that the type of the personal information of the first user is the second type, perform a request process of requesting the first information system to give a permission for sending the personal information of the first user from the first information server. The control unit may perform the first sending process on a condition that the permission is received from the first information system. The information management device of this aspect differs the requirement or non-requirement for the permission of transmission of the personal information according to the type of the personal information. This configuration improves the convenience and the flexibility in handling the personal information.

(9) In the information management device of the above aspect may further comprises: a memory unit configured to store a first table that is configured to specify a correlation of each of the plurality of information systems to each of multiple different types of the personal information. The control unit may be configured to, when receiving the approval of the first user for the second information system, perform an identification process of identifying the type of the personal information as a subject of the approval. The control unit may perform the first sending process on a condition that the identified type of the personal information is correlated to the second information system by the first table. In the process of sending the personal information, the information management device of this aspect causes only the type of the personal information correlated to the information system as a sending destination to be sent selectively. This configuration improves the convenience in handling the personal information.

(10) In the information management device of the above aspect, the first table may be configured to specify the correlation of each of the plurality of information systems to each of the multiple different types of the personal information by specifying a correlation of each of multiple different types of the information systems to each of the multiple different types of the personal information. In the process of sending the personal information, the information management device of this aspect causes only the type of the personal information correlated to the type of the information system as a sending destination to be sent selectively. This configuration does not require to individually store the correlation of the individual information systems to the types of personal information and further improves the convenience in handling the personal information.

(11) In the information management device of the above aspect may further comprises: a memory unit configured to store a second table that is configured to specify a correlation of each of a plurality of the storage servers to each of a plurality of the users. The control unit may send the personal information of the first user to the storage server correlated to the first user by the second table in the first sending process. The information management device of this aspect enables the personal information of the respective users to be stored in a plurality of storage servers dispersedly. This configuration reduces the volume of the personal information stored in each storage server and achieves load distribution and reduction of the leakage risk.

(12) In the information management device of the above aspect may further comprises: a memory unit configured to store a third table that is configured to specify a correlation of each of a plurality of the storage servers to each of multiple different types of the personal information. The control unit may send the personal information of the first user to the storage server correlated to the type of the personal information of the first user by the third table in the first sending process. The information management device of this aspect enables various types of personal information to be stored in a plurality of storage servers dispersedly. This configuration reduces the volume of the personal information stored in each storage server and achieves load distribution and reduction of the leakage risk.

(13) In the information management device of the above aspect, the personal information may include medical information. The information management device of this aspect causes the medical information that is especially of significance in accumulation to be securely handed over to the second information server. This configuration accordingly enables the medical information to be effectively used by the second information system.

(14) In the information management device of the above aspect, each of the information systems may allow a plurality of members to use the information server. The control unit may receive approval or disapproval for each of the members in the receiving process. When receiving the disapproval for all the members allowed to use the first information server, the control unit may determine that the disapproval for the first information system is received. When receiving the approval for at least one of the members allowed to use the second information server, the control unit may determine that the approval for the second information system is received. The information management device of this aspect receives approval or disapproval for each member and thereby receives approval or disapproval for the corresponding information system.

(15) In the information management device of the above aspect, the plurality of information systems may include: a fifth information system configured to generate and use specific personal information that is a specific type of the personal information, and a sixth information system configured to use the specific personal information without generating the specific personal information. The control unit may be configured to, when receiving approval of a fourth user for the sixth information system to use the specific personal information of the fourth user generated by the fifth information system, perform a fourth sending process of causing the specific personal information of the fourth user stored in a fifth information server provided in the fifth information system to be sent from the fifth information server to the storage server and to be stored into the storage server, and causing the specific personal information of the fourth user stored in the storage server to be sent from the storage server to a sixth information server provided in the sixth information system and to be stored into the sixth information server. The information management device of this aspect enables the personal information generated by the information system generating and using the personal information to be handed over to and used by the information system using the personal information without generating the personal information. This configuration ensures the effective use of the personal information.

(16) In the information management device of the above aspect, the personal information may include at least one piece of attribute information indicating an attribute of each user. The approval received in the receiving process may be either full approval that is approval for use of the personal information including all the attribute information or partial approval that is approval for use of partial personal information excluding at least one piece of the attribute information from the personal information. The control unit may be configured to, when receiving partial approval of a fifth user for a seventh information system that is one of the plurality of information systems to use the partial personal information of the fifth user, perform a fifth sending process of causing the partial personal information out of the personal information of the fifth user stored in an eighth information server provided in an eight information system that is one of the plurality of information systems to be sent from the eighth information server to the storage server and to be stored into the storage server, and causing the partial personal information of the fifth user stored in the storage server to be sent from the storage server to a seventh information server provided in the seventh information system and to be stored into the seventh information server. The information management device of this aspect provides an option of approval for the use of the partial personal information excluding at least one piece of the attribute information from the personal information, in addition to an option of approval for the use of the entire personal information, as the possible options of approval for the use of the personal information. This configuration accelerates the effective use of the personal information, while protecting the privacy of the user.

(17) In the information management device of the above aspect, the control unit may be configured to, when receiving approval of a sixth user for two or more subject information systems that are included in the plurality of information systems and are other than a ninth information system that is one of the plurality of information systems, to use the personal information of the sixth user that is stored in a ninth information server provided in the ninth information system in one cycle of the receiving process, perform a sixth sending process of causing the personal information of the sixth user stored in the ninth information server to be sent from the ninth information server to the storage server and to be stored into the storage server, and causing the personal information of the sixth user stored in the storage server to be sent from the storage server to information servers respectively provided in the two or more subject information systems and to be stored into the information servers. The information management device of this aspect enables the approval process for the user of the personal information and the transfer process of the personal information by the two or more subject information systems to be performed efficiently.

(18) An information management system disclosed in the description hereof may comprise a storage server; information servers respectively provided in a plurality of information systems; and the information management device described above. Each of the information servers may comprise a system communication unit; and a system control unit. The control unit may receive approval or disapproval for each member in the receiving process and send approval information indicating either the approval or the disapproval to a corresponding information system. The system control unit may be configured to: perform an information receiving process of receiving the approval information via the system communication unit; and when receiving the approval information, perform a setting process of setting approval or disapproval for each member to use the personal information of the user. In the information management system of this aspect, each of the information servers sets approval or disapproval for each member to use the personal information of the user, in response to approval or disapproval expressed by the user.

The technique disclosed in the description hereof may be implemented by various aspects, for example, the information management device, the information management system including the information management device, an information management method, computer programs that implement the method, the device or the functions of the system, and non-transitory recording media in which such computer programs are recorded.

### BRIEF DESCRIPTION

Some of the embodiments will be described in detail, with references to the following figures, wherein like designations denote like members, wherein:

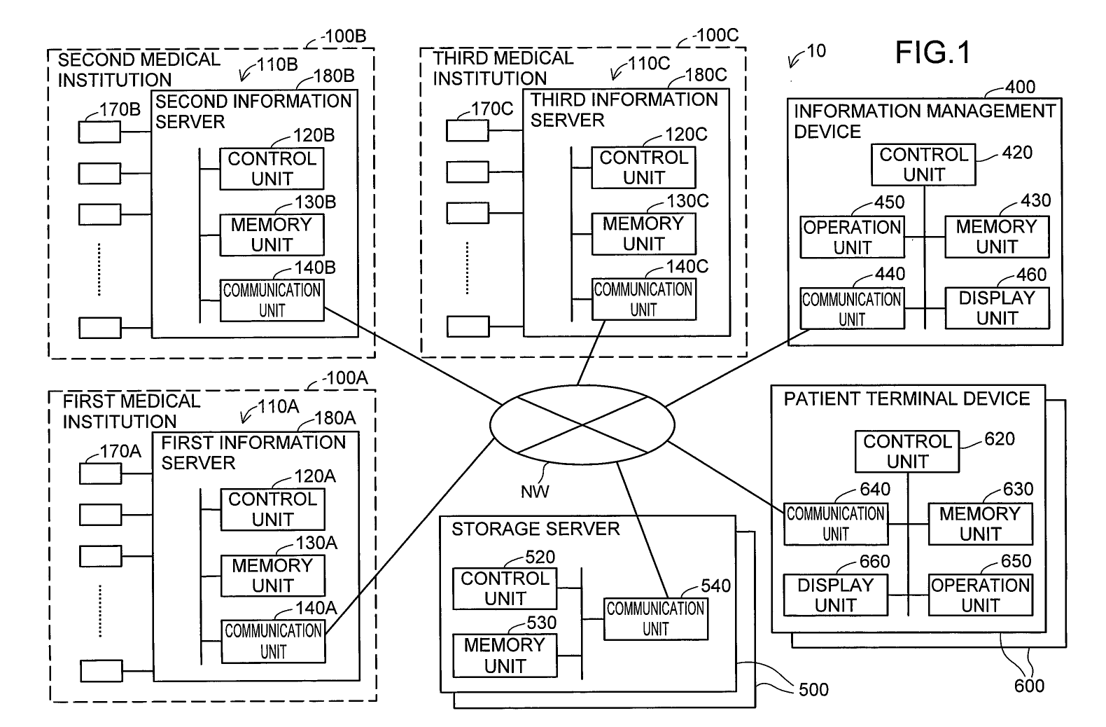

FIG. 1 is a diagram illustrating the configuration of an information management system, in accordance with embodiments of the present invention;

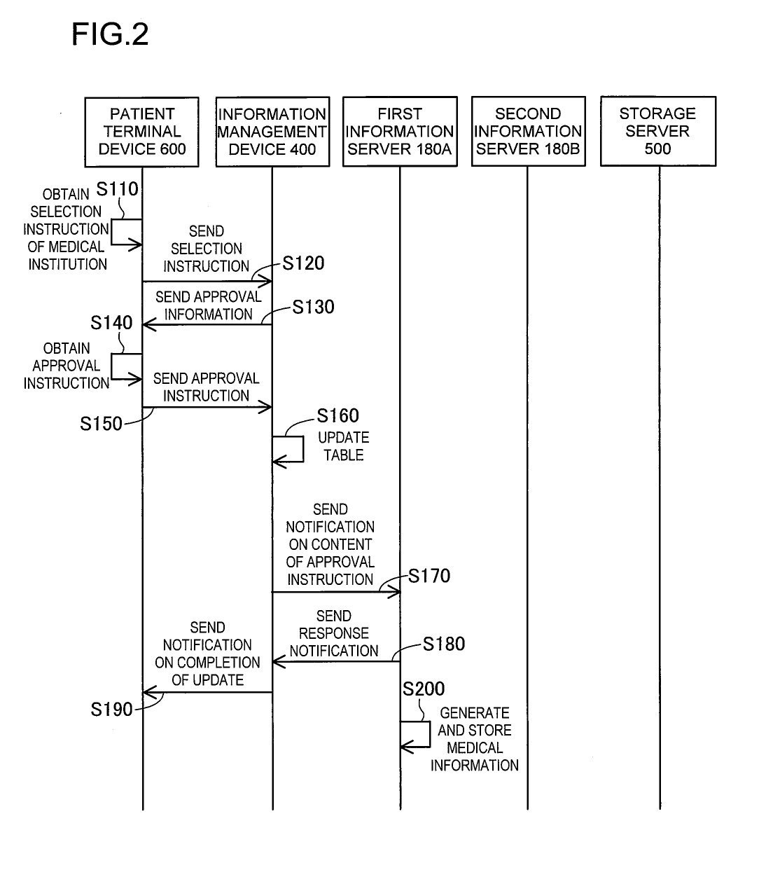

FIG. 2 is a diagram showing a flow chart of a personal information management process in the information management system, in accordance with embodiments of the present invention;

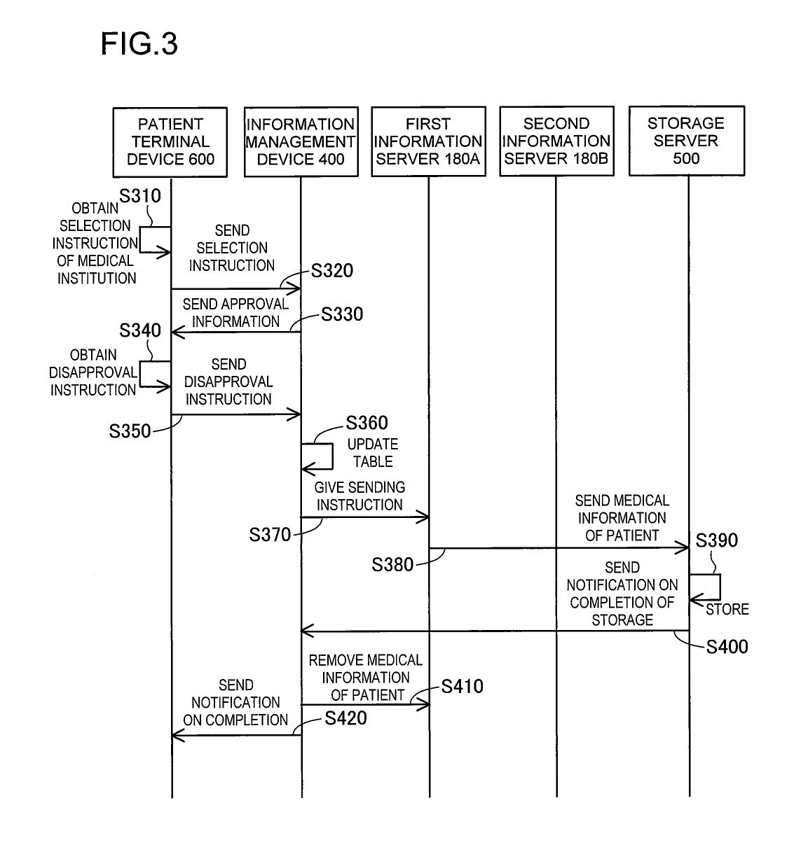

FIG. 3 is a diagram showing another flow chart of the personal information management process in the information management system, in accordance with embodiments of the present invention;

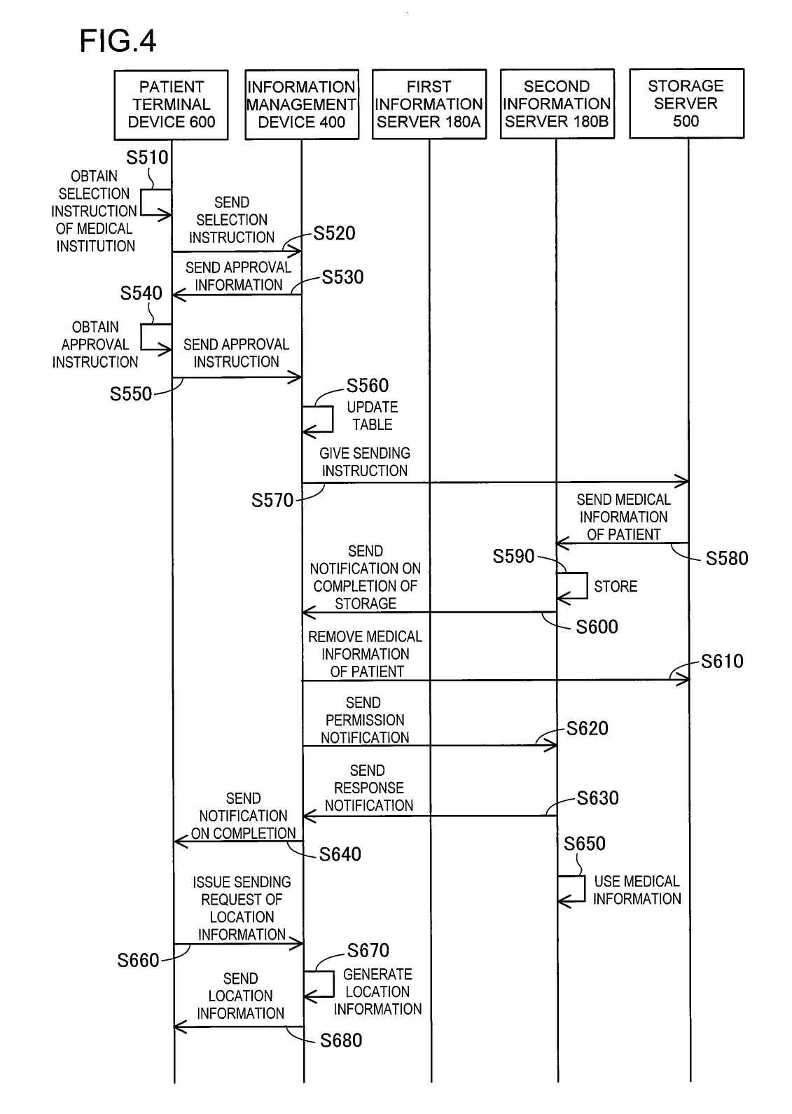

FIG. 4 is a diagram showing another flow chart of the personal information management process in the information management system, in accordance with embodiments of the present invention;

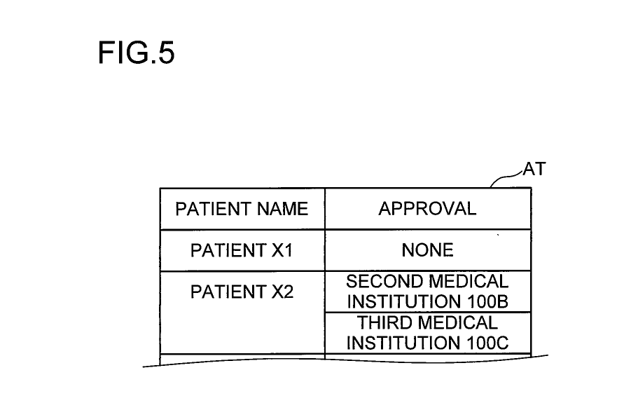

FIG. 5 is a diagram showing one example of an approval information table, in accordance with embodiments of the present invention;

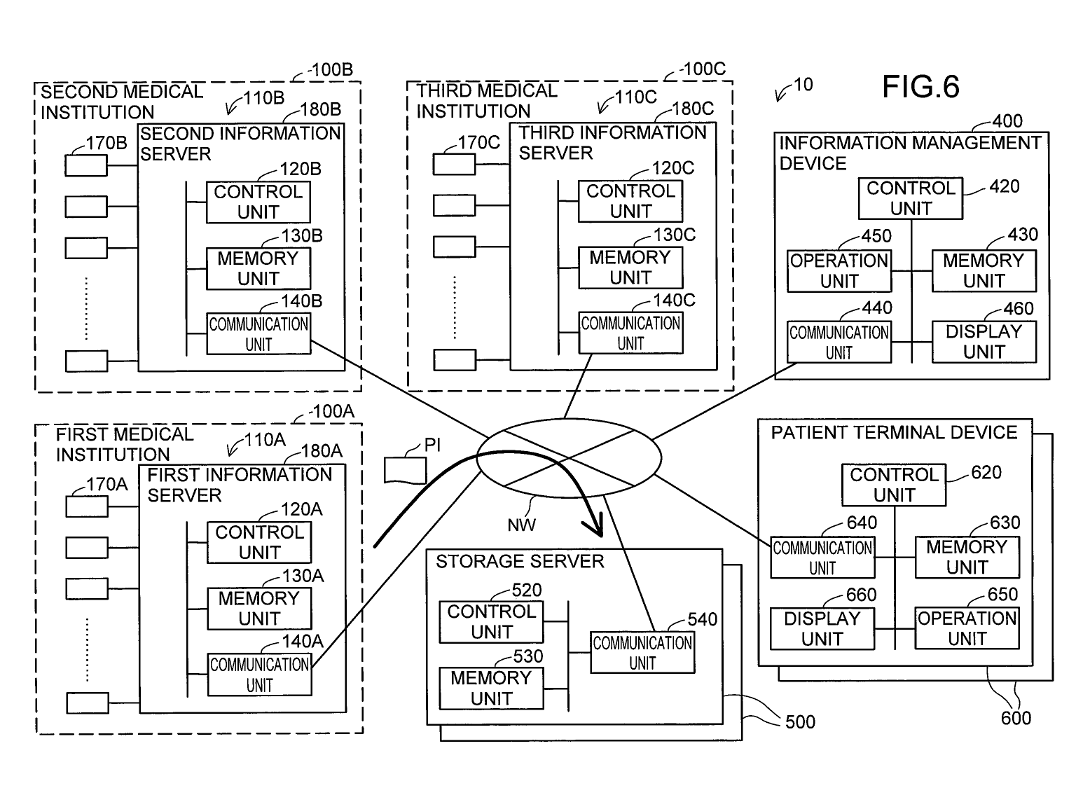

FIG. 6 is a diagram illustrating a process of sending medical information of a patient from a first information server to a storage server, in accordance with embodiments of the present invention;

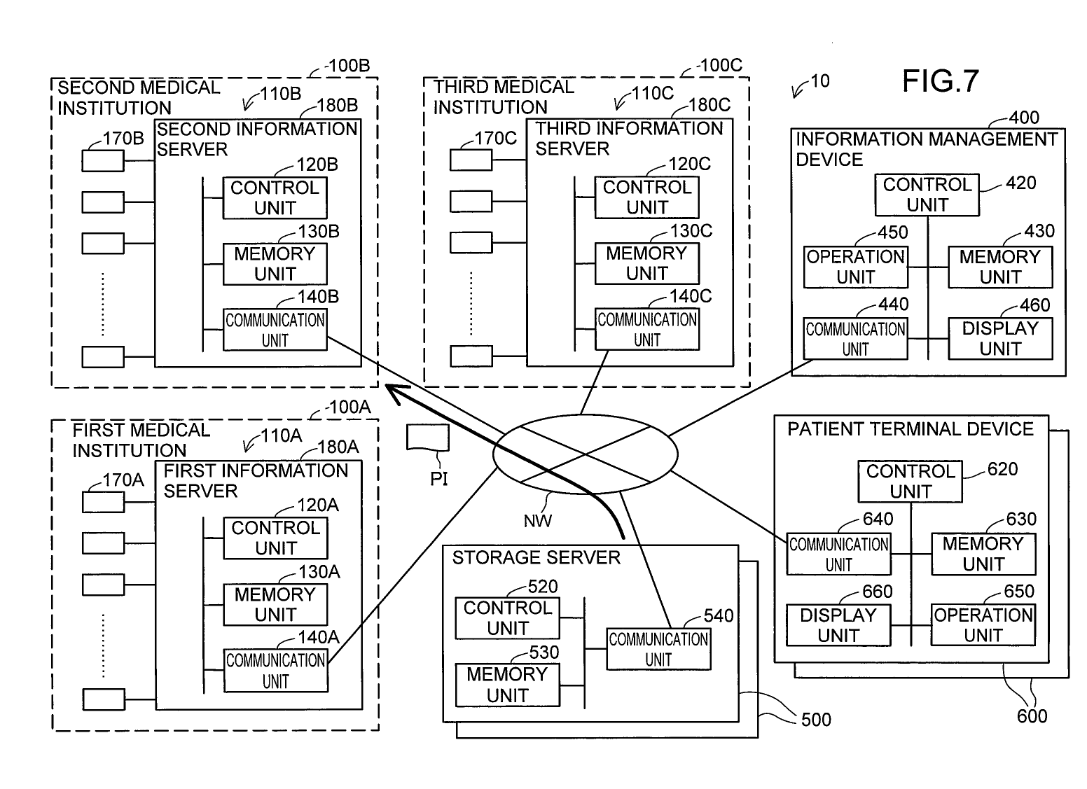

FIG. 7 is a diagram illustrating a process of sending the medical information of the patient from the storage server to a second information server, in accordance with embodiments of the present invention;

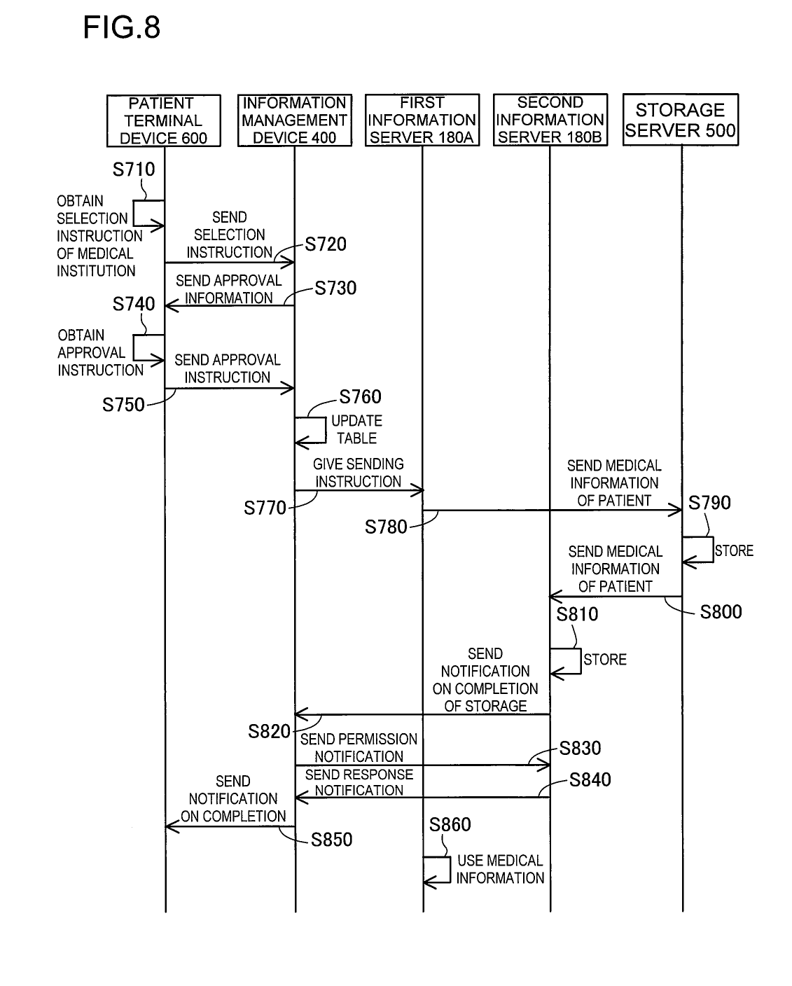

FIG. 8 is a sequence diagram showing a personal information management process according to a first modification;

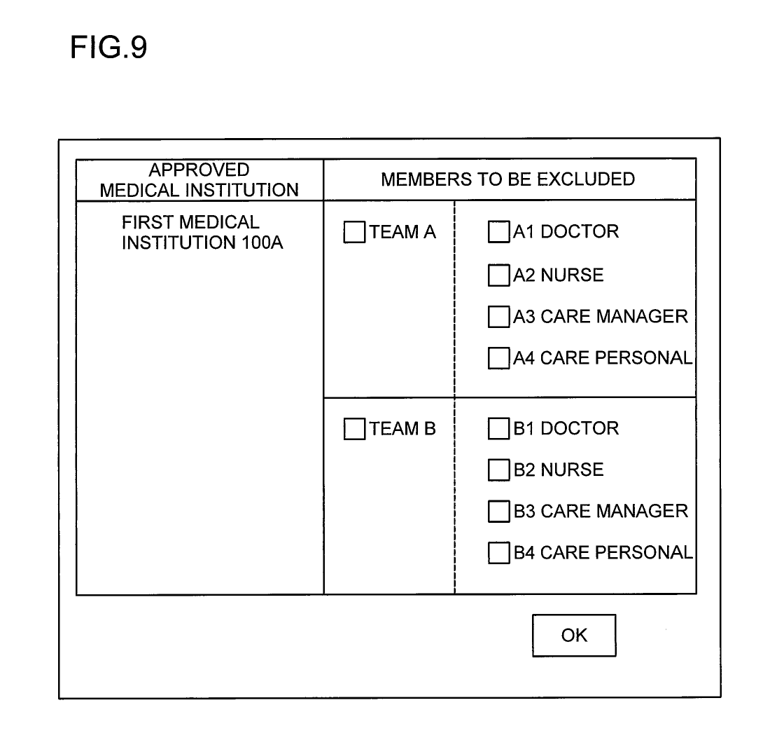

FIG. 9 is a diagram illustrating one example of a user interface according to a second modification;

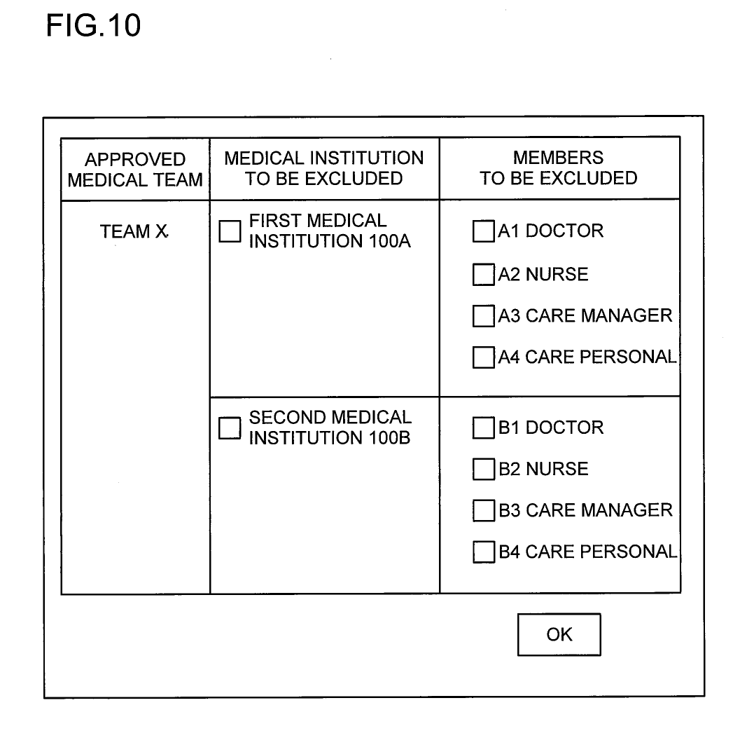

FIG. 10 is a diagram illustrating one example of a user interface according to a third modification;

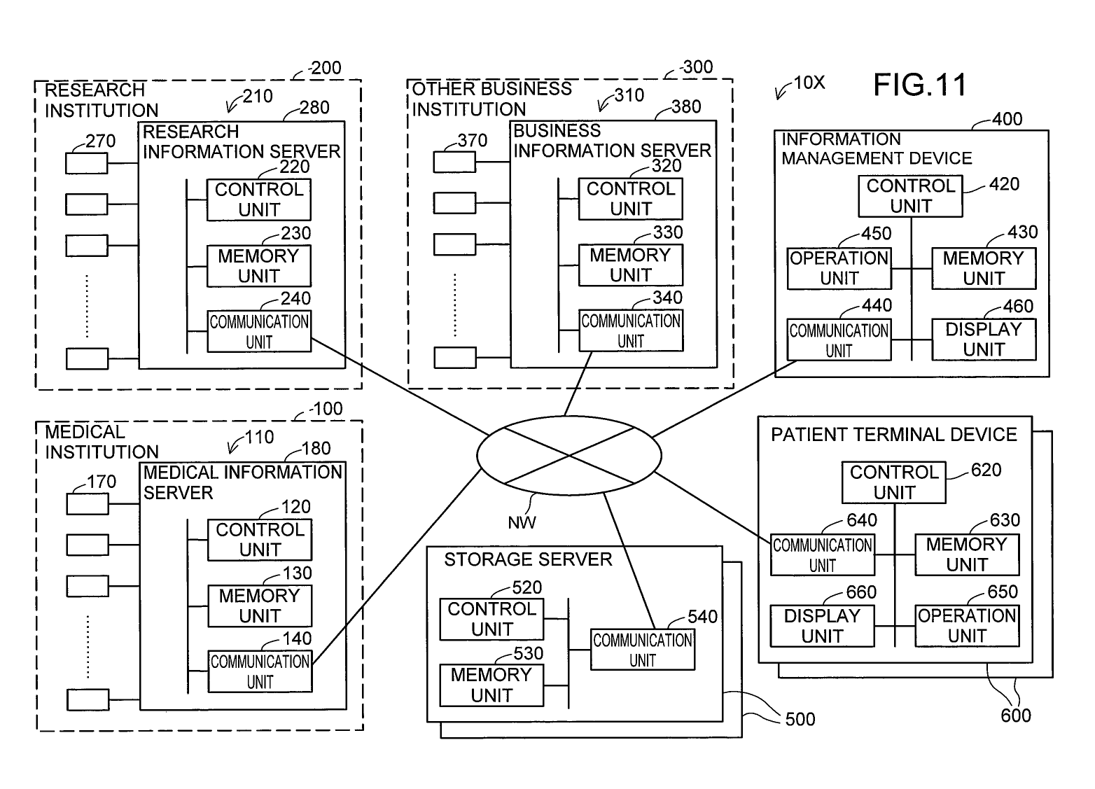

FIG. 11 is a diagram illustrating the configuration of an information management system according to a fourth modification;

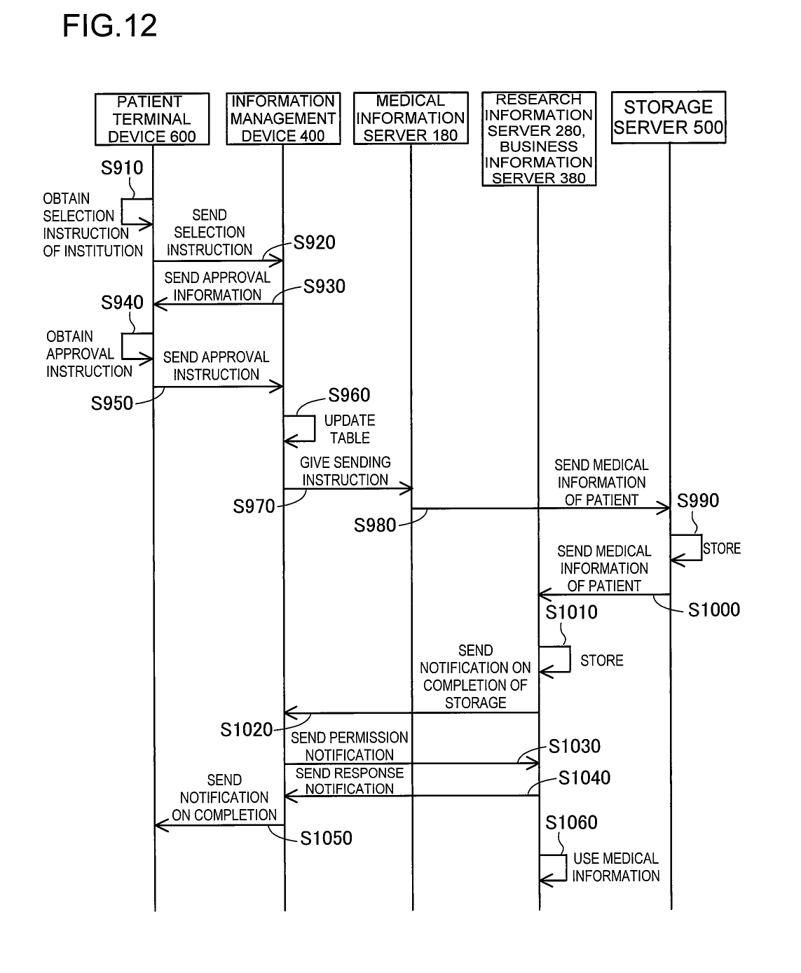

FIG. 12 is a diagram showing the flow of a personal information management process in the information management system according to the fourth modification; and

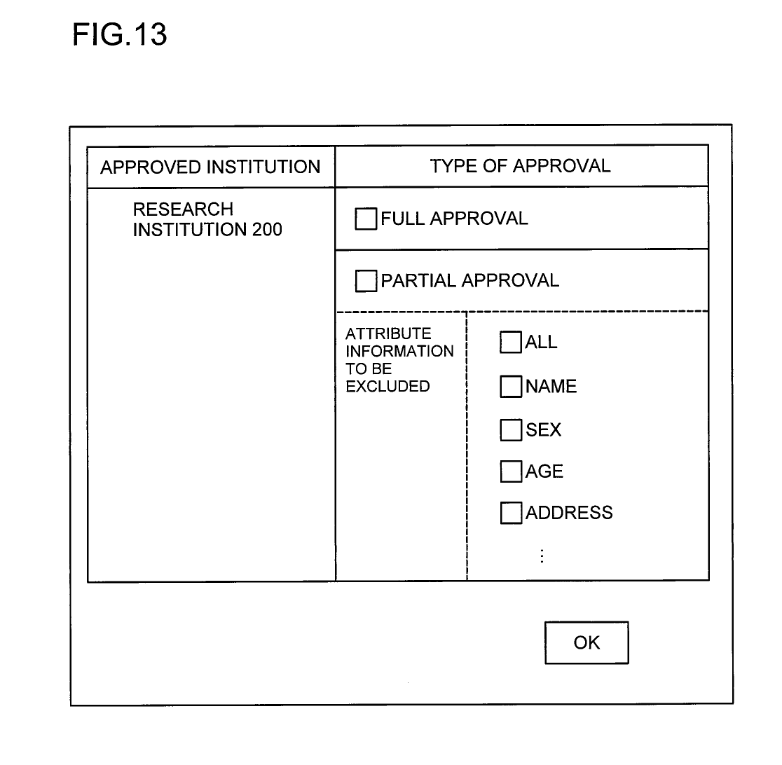

FIG. 13 is a diagram illustrating one example of a user interface according to the fourth modification.

### DETAILED DESCRIPTION

FIG. 1 is a diagram illustrating the configuration of an information management system 10. The information management system 10 includes a plurality of information systems 110 (first information system 110A, second information system 110B and third information system 110C) respectively operated by a plurality of medical institutions 100 (first medical institution 100A, second medical institution 100B and third medical institution 100C), an information management device 400, a plurality of storage servers 500, and a plurality of patient terminal devices 600. The respective devices and systems are interconnected via a network NW. The medical institution 100 is a facility, such as a hospital, a medical office, a clinic, a home-visit nursing station, a care support office, or a nursing-care service office. FIG. 1 illustrates three medical institutions 100, but the number of the medical institutions 100 included in the information management system 10 may be two or may be four or more. Similarly, FIG. 1 illustrates the two storage servers 500 and two patient terminal devices 600, but the number of the storage servers 500 and the number of the patient terminal devices 600 included in the information management system 10 may be one or may be three or more. In the description below, when there is a need to individually distinguish the respective medical institutes 100 and components of the respective medical institutions 100 from one another, ordinal numbers such as “first” are prefixed to the names of the respective medical institutions and their components for the purpose of identifying the respective medical institutions, and alphabetical codes such as “A” are suffixed to the reference signs of the respective medical institutions and their components for the purpose of identifying the respective medical institutions. These ordinal numbers and alphabetical codes are omitted appropriately when the description is common to all the medical institutions 100.

The information system 110 operated by the medical institution 100 is a computer system configured to record medical information such as results of physical examinations and results of medical tests generated by respective doctors, respective staff members and the like belonging to the medical institution 100 and to allow the respective doctors, the respective staff members and the like to access the medical information as needed basis, so as to support the works of the respective doctors, the respective staff members and the like. The information system 110 includes a plurality of terminal devices 170 used by the respective doctors, the respective staff members and the like, and one or a plurality of information servers 180 connected with the respective terminal devices 170 via a medical institution intranet. The terminal devices 170 used may be, for example, personal computers, tablet terminals and smartphones.

The information server 180 includes a control unit 120, a memory unit 130 and a communication unit 140. The memory unit 130 is configured by, for example, a hard disk drive (hereinafter referred to as “HDD”), a ROM, a RAM or the like to store various data such as medical information of patients, various programs used to control the information system 110, and the like. The communication unit 140 is an interface configured to make communication with external devices by a wireless communication system or a wired communication system. The control unit 120 is configured by, for example, a central processing unit (hereinafter referred to as “CPU”) or the like to control the respective components of the information system 110 according to programs read from the memory unit 130.

The information management device 400 is a device configured to manage medical information of patients stored in the memory units 130 of the respective information servers 180. The information management device 400 includes a control unit 420, a memory unit 430, a communication unit 440, an operation unit 450 and a display unit 460. The memory unit 430 is configured by, for example, an HDD, a ROM, a RAM or the like to store various data such as medical information of patients and various programs used to control the information management device 400, for example, a program used to perform a personal information management process described later. The communication unit 440 is an interface configured to make communication with external devices by a wireless communication system or a wired communication system. The operation unit 450 is configured by, for example, a keyboard, a mouse or the like to receive an administrator's operations. The display unit 460 is configured by, for example, a liquid crystal display or the like. The control unit 420 is configured by, for example, a CPU or the like to control the respective components of the information management device 400 according to programs read from the memory unit 430.

The storage server 500 is a device configured to store medical information of patients temporarily. The storage server 500 includes a control unit 520, a memory unit 530 and a communication unit 540. The memory unit 530 is configured by, for example, an HDD, a ROM, a RAM or the like to store various data such as medical information of patients and various programs used to control the storage server 500. The communication unit 540 is an interface configured to make communication with external devices by a wireless communication system or a wired communication system. The control unit 520 is configured by, for example, a CPU or the like to control the respective components of the storage server 500 according to programs read from the memory unit 530.

The patient terminal device 600 is a device used by each patient and may be, for example, a personal computer, a tablet terminal or a smartphone. The patient terminal device 600 may be placed, for example, at each patient's home or in each medical institution 100. The patient terminal device 600 includes a control unit 620, a memory unit 630, a communication unit 640, an operation unit 650 and a display unit 660. The memory unit 630 is configured by, for example, an HDD, a ROM, a RAM or the like to store various data and various programs used to control the patient terminal device 600. The communication unit 640 is an interface configured to make communication with external devices by a wireless communication system or a wired communication system. The operation unit 650 is configured by, for example, a keyboard, a mouse or the like to receive each patient's operations. The display unit 660 is configured by, for example, a liquid crystal display or the like. The control unit 620 is configured by, for example, a CPU or the like to control the respective components of the patient terminal device 600 according to programs read from the memory unit 630.

### A-2. Personal Information Management Process

FIGS. 2 to 4 are diagrams showing flows of a personal information management process in the information management system 10 according to an embodiment. The personal information management process is a process of, for example, sending and removing medical information of each patient in response to receiving approval or disapproval of the patient for each medical institution 100 (or each information system 110) to use the medical information of the patient (hereinafter may be expressed as “approval or disapproval for the information system 110” as a matter of convenience). The disapproval herein includes expressing a disapproval intention, in addition to withdrawal of the approval. The following describes some examples of the personal information management process performed in a situation where a patient X1 intends to visit, for example, the first medical institution 100A and gives approval for the first information system 110A operated by the first medical institution 100A (situation 1), in a situation where the patient X1 subsequently intends to change the visiting medical institution from the first medical institution 100A to the second medical institution 100B because of a move or the like and withdraws the approval for the first information system 110A (expresses a disapproval intention) (situation 2), and in a situation where the patient X1 gives approval for the second information system 110B operated by the second medical institution 100B (situation 3).

FIG. 2 is a sequence diagram showing the personal information management process performed in the situation 1 where the patient X1 gives approval for the first information system 110A. The control unit 620 of the patient terminal device 600 first authenticates the patient X1 by a known authentication system, subsequently causes a user interface for selecting a medical institution 100 that is a subject of approval or disapproval to be displayed on the display unit 660, and obtains a selection instruction for selecting one or a plurality of medical institutions 100 via the operation unit 650 (S110). It is here assumed that the control unit 620 obtains a selection instruction for selecting the first medical institution 100A. The control unit 620 of the patient terminal device 600 sends the obtained selection instruction to the information management device 400 (S120).

When receiving the selection instruction sent from the patient terminal device 600, the control unit 420 of the information management device 400 sends approval information indicating an approval status of the patient X1 with respect to the medical institution 100 (first medical institution 100A) selected by the selection instruction, to the patient terminal device 600 (S130). An approval information table AT showing approval statuses of the respective patients is stored in the memory unit 430 of the information management device 400. FIG. 5 is a diagram illustrating one example of the approval information table AT. The approval information table AT includes information used to identify each medical institution 100 selected by each patient to give approval for the use of the patient's medical information. In the illustrated example of FIG. 5, the patient X1 does not give approval for any medical institutions 100, and a patient X2 gives approval for the second medical institution 100B and the third medical institution 100C. In this illustrated example, the control unit 420 of the information management device 400 sends approval information showing that the patient X1 does not give approval for the first medical institution 100A, to the patient terminal device 600. The control unit 620 of the patient terminal device 600 receives the approval information sent from the information management device 400 and causes information showing that the patient X1 does not give approval for the first medical institution 100A to be displayed on the display unit 660. This enables the patient X1 to confirm that the patient X1 does not give approval for the first medical institution 100A.

The control unit 620 of the patient terminal device 600 causes a user interface for giving approval or disapproval for the information system 110 operated by the medical institution 100 selected at S110, to be displayed on the display unit 660, and obtains an approval instruction or a disapproval instruction via the operation unit 650 (S140). It is here assumed that the patient X1 enters an approval instruction for the first information system 110A operated by the first medical institution 100A. The control unit 620 of the patient terminal device 600 sends the obtained approval instruction to the information management device 400 (S150).

The control unit 420 of the information management device 400 receives the approval instruction sent from the patient terminal device 600 and updates the approval information table AT, based on the received approval instruction (S160). In this illustrated example, the approval of the patient X1 for the first medical institution 100A is registered in the approval information table AT (shown in FIG. 5).

The control unit 420 of the information management device 400 subsequently sends a notification on the content of the approval instruction to the information server 180 provided in the information system 110 that is the subject of the approval instruction (S170). In this illustrated example, the notification on the approval of the patient X1 is sent to the first information server 180A provided in the first information system 110A.

When receiving the notification sent from the information management device 400, the control unit 120A of the first information server 180A sends a response notification to the information management device 400 (S180). When receiving the response notification sent from the information server 180, the control unit 420 of the information management device 400 sends a notification on completion of update to the patient terminal device 600 (S190). The control unit 620 of the patient terminal device 600 receives the notification on completion of update sent from the information management device 400 and causes information indicating completion of the approval process by the patient X1 for the first medical institution 100A to be displayed on the display unit 660. This enables the patient X1 to confirm completion of the approval process for the first medical institution 100A. After that, the first information system 110A allows each of the doctors, the staff members and the like belonging to the first medical institution 100A to generate and store medical information as the results of physical examinations and medical tests of the patient X1 into the first information server 180A and to access the medical information (S200).

FIG. 3 is a sequence diagram showing the personal information management process performed in the situation 2 where the patient X1 makes disapproval for the first information system 110A. The processing details of S310 to S360 in FIG. 3 are similar to the processing details of S110 to S160 in FIG. 2 and are not specifically described here. FIG. 3, however, describes the process in the situation 2 where the patient X1 makes disapproval for the first information system 110A. The process accordingly obtains a selection instruction for selecting the first medical institution 100A at S310, sends the selection instruction at S320, sends approval information on the first medical institution 100A at S330, obtains a disapproval instruction (withdrawal of the approval) for the first information system 110A operated by the first medical institution 100A at S340, sends the disapproval instruction at S350, and registers the disapproval of the patient X1 for the first medical institution 100A into the approval information table AT (shown in FIG. 5) at S360.

The control unit 420 of the information management device 400 gives the information server 180 provided in the information system 110 that is the subject of the disapproval instruction, an instruction to send medical information to the storage server 500 (S370). In this illustrated example, an instruction to send the medical information of the patient X1 to the storage server 500 is given to the first information server 180A provided in the first information system 110A. According to the embodiment, the information management system 10 includes the plurality of storage servers 500, and a table correlating the individual patients to the respective storage servers 500 is stored in the memory unit 430 of the information management device 400. The control unit 420 of the information management device 400 gives the first information server 180A an instruction to identify the storage server 500 correlated to the patient X1 by referring to this table and to send the medical information to the identified storage server 500.

When receiving this sending instruction sent from the information management device 400, the control unit 120A of the first information server 180A sends the medical information of the patient X1 to the specified storage server 500 (S380). The control unit 520 of the storage server 500 stores the medical information of the patient X1 sent from the first information server 180A into the memory unit 530 (S390) and sends a notification on completion of storage of the medical information of the patient X1 to the information management device 400 (S400). FIG. 6 illustrates a process of sending medical information P1 of the patient X1 from the first information server 180A to the storage server 500.

The control unit 420 of the information management device 400 receives the notification on completion of storage sent from the storage server 500 and accesses the first information system 110A to remove the medical information of the patient X1 from the first information server 180A (S410). The medical information of the patient X1 is accordingly removed from the first information server 180A and is present only in the storage server 500. The control unit 420 of the information management device 400 sends a notification on completion of sending and removing the medical information, to the patient terminal device 600 of the patient X1 (S420). This enables the patient X1 to confirm completion of the disapproval process for the first medical institution 100A.

FIG. 4 is a sequence diagram showing the personal information management process performed in the situation 3 where the patient X1 gives approval for the second information system 100B after the patient X1 makes disapproval for the first information system 110A. The processing details of S510 to S560 in FIG. 4 are similar to the processing details of S110 to S160 in FIG. 2 and are not specifically described here. FIG. 4, however, describes the process in the situation 3 where the patient X1 gives approval for the second information system 110B. The process accordingly obtains a selection instruction for selecting the second medical institution 100B at S510, sends the selection instruction at S520, sends approval information on the second medical institution 100B at S530, obtains an approval instruction for the second information system 110B operated by the second medical institution 100B at S540, sends the approval instruction at S550, and registers the approval of the patient X1 for the second medical institution 100B into the approval information table AT (shown in FIG. 5) at S560.

The control unit 420 of the information management device 400 gives the storage server 500 that stores the medical information of the patient X1, an instruction to send the medical information to the information server 180 provided in the information system 110 that is the subject of the approval instruction (S570). In this illustrated example, an instruction to send the medical information of the patient X1 to the second information server 180B provided in the second information system 110B is given to the storage server 500.

When receiving the sending instruction sent from the information management device 400, the control unit 520 of the storage server 500 sends the medical information of the patient X1 to the second information server 180B (S580). The control unit 120B of the second information server 180B stores the medical information of the patient X1 sent from the storage server 500, into the memory unit 130B (S590) and sends a notification on completion of storage of the medical information of the patient X1 to the information management device 400 (S600). FIG. 7 illustrates a process of sending the medical information P1 of the patient X1 from the storage server 500 to the second information server 180B.

The control unit 420 of the information management device 400 receives the notification on completion of storage sent from the second information server 180B and accesses the storage server 500 to remove the medical information of the patient X1 from the storage server 500 (S610). The medical information of the patient X1 is accordingly removed from the storage server 500 and is present only in the second information server 180B.

The control unit 420 of the information management device 400 sends a permission notification for the use of the medical information of the patient X1, to the second information server 180B (S620). When receiving the permission notification, the control unit 120B of the second information server 180B sends a response notification to the information management server 400 (S630). When receiving the response notification, the control unit 420 of the information management device 400 sends a notification on completion of sending and removing the medical information, to the patient terminal device 600 of the patient X1 (S640). This enables the patient X1 to confirm completion of the approval process for the second medical institution 100B. After that, the second information system 110B allows each of the doctors, the staff members and the like belonging to the second medical institution 100B to access and use the medical information of the patient X1 handed over from the first information system 110A and to newly generate and store medical information as the results of physical examinations and medical tests of the patient X1 into the second information server 180B and to access the medical information (S650).

The control unit 620 of the patient terminal device 600 may cause a user interface for issuing a sending request of location information that shows a server where medical information of each patient is stored, to be displayed on the display unit 660. The control unit 620 of the patient terminal device 600 obtains an instruction to issue a sending request of location information via the operation unit 650 and issues a sending request of location information to the information management device 400 (S660). When receiving the sending request, the control unit 420 of the information management device 400 generates location information showing a server where the medical information of the patient X1 is stored (S670) and sends the generated location information to the patient terminal device 600 of the patient X1 (S680). The patient terminal device 600 receives the location information and displays the received location information onto the display unit 660. This enables the patient X1 to check the location of the own medical information. In the illustrated example of FIG. 4, location information showing that the medical information of the patient X1 is stored in the second information server 180B is generated and sent.

As described above, when receiving disapproval for the first information system 110A, the information management system 10 of the embodiment causes the medical information of the patient X1 that is stored in the first information server 180A provided in the first information system 110A to be sent from the first information server 180A to the storage server 500 and to be stored into the storage server 500. When receiving approval for the second information system 110B, the information management system 10 of the embodiment causes the medical information of the patient X1 stored in the storage server 500 to be sent from the storage server 500 to the second information server 180B provided in the second information system 110B and to be stored into the second information server 180B. This means that the medical information of the patient X1 stored in the first information server 180A is once stored into the storage server 500 before being handed over to the second information server 180B. The information management system 10 of the embodiment suppresses the occurrence of an event that the medical information of the patient X1 disappears in the process of handing over the medical information of the patient X1 stored in the first information server 180A to the second information server 180B. Accordingly, the information management system 10 of the embodiment enables the medical information of the patient X1 to be securely handed over from the first information server 180A to the second information server 180B.

Especially the medical information is of great significance in accumulation and is significantly affected by removal. The information management system 10 of this embodiment causes the medical information of the patient X1 to be securely handed over to the second information server 180B and thereby enables the medical information of the patient X1 generated by the first information system 110A to be effectively used by the second medical institution 100B.

The storage server 500 is provided independently of the respective medical institutions 100. Consequently, for example, even when the respective medical institutions 100 employ the operation of uniformly removing the medical information after elapse of a predetermined storage period, the storage server 500 can store the medical information regardless of this storage period and enables the medical information of each patient to be stored for the patient's desired time period and to be effectively used.

When receiving disapproval for the first information system 110A, the information management system 10 of the embodiment causes the medical information of the patient X1 to be sent from the first information server 180A to the storage server 500 and subsequently removes the medical information of the patient X1 from the first information server 180A. This configuration securely removes the medical information of the patient X1 from the first information sever 180A that is the subject of disapproval of the patient X1 and prevents the medical information of the patient X1 from being used by the first medical institution 100A without approval. Additionally, the first information system 110A is not required to manage whether the medical information of the patient X1 is removed or not from the first information server 180A. This configuration reduces the process load of the first information system 110A.

When receiving approval for the second information system 110B, the information management system 10 of the embodiment causes the medical information of the patient X1 to be sent from the storage server 500 to the second information server 180B and subsequently removes the medical information of the patient X1 from the storage server 500. This configuration securely removes the medical information of the patient X1 from the storage server 500 and minimizes the risk of leakage of the medical information of the patient X1.

The information management system 10 of the embodiment receives approval or disapproval of the patient X1 for each information system 110 via the network NW. This configuration simplifies and accelerates the process of receiving approval or disapproval, compared with a conventional method of receiving approval or disapproval in writing.

When receiving a sending request of location information from the patient terminal device 600, the information management system 10 of the embodiment sends location information for identifying the server where the medical information of the patient is stored, to the patient terminal device 600. The patient is thus allowed to check the location of the own medical information by referring to the location information and to recognize which medical institution 100 uses the own medical information.

Furthermore, in the information management system 10 of the embodiment, the individual patients are correlated to the respective storage servers 500, and the medical information of each patient is sent to the storage server 500 that is correlated to the patient. The information management system 10 of the embodiment accordingly enables the medical information of the respective patients to be stored dispersedly in the plurality of storage servers 500. This configuration reduces the volume of medical information stored in each of the storage servers 500 and achieves load distribution and reduction of the leakage risk.

### B. Modifications

The technique disclosed in the description hereof is not limited to the above embodiment but may be modified to various aspects without departing from the scope of the disclosure. Some of possible modifications are given below.

### B-1. First Modification

The above embodiment describes the example where the patient X1 gives approval for the second information system 110B while making disapproval for the first information system 110A, for example, as in the case of move of the patient X1. In another example, as in the case where the patient X1 needs a second opinion, the patient X1 may give approval for the second information system 110B while not making disapproval for the first information system 110A. The following describes the personal information management process performed in such a case.

FIG. 8 is a sequence diagram showing the personal information management process performed in a situation where the patient X1 gives approval for the second information system 110B while maintaining approval for the first information system 110A. The processing details of S710 to S760 in FIG. 8 are similar to the processing details of S510 to S560 in FIG. 4 and are not specifically described here.

The control unit 420 of the information management device 400 gives the first information server 180A provided in the first information system 110A, an instruction to send the medical information to the storage server 500 (S770). When receiving the sending instruction, the control unit 120A of the first information server 180A sends the medical information of the patient X1 to the specified storage server 500 (S780). The control unit 520 of the storage server 500 stores the received medical information of the patient X1 into the memory unit 530 (S790). The control unit 520 of the storage server 500 subsequently sends the medical information of the patient X1 to the second information server 180B (S800). The control unit 120B of the second information server 180B stores the received medical information of the patient X1 into the memory unit 130B (S810) and sends a notification on completion of storage of the medical information of the patient X1 to the information management device 400 (S820).

When receiving the notification on completion of storage, the control unit 420 of the information management device 400 sends a permission notification for the use of the medical information of the patient X, to the second information server 180B (S830). When receiving the permission notification, the control unit 120B of the second information server 180B sends a response notification to the information management device 400 (S840). When receiving the response notification, the control unit 420 of the information management device 400 sends a notification on completion of sending the medical information to the patient terminal device 600 of the patient X1 (S850). This enables the patient X1 to confirm completion of the approval process for the second medical institution 100B. After that, the second information system 110B allows each of the doctors, the staff members and the like belonging to the second medical institution 100B to access and use the medical information of the patient X1 handed over from the first information system 110A (S860).

As described above, the first modification shown in FIG. 8 receives approval of the patient X1 for the first information system 110A and causes the medical information of the patient X1 to be stored into the first information server 180A provided in the first information system 110A. The first modification subsequently receives approval of the patient X1 for the second information system 110B and causes the medical information of the patient X1 stored in the first information server 180A to be sent from the first information server 180A to the storage server 500, to be further sent from the storage server 500 to the second information server 180B provided in the second information system 110B and to be stored into the second information server 180B. The first modification shown in FIG. 8 accordingly enables medical information of one identical patient to be readily stored into respective information servers 180 of a plurality of information systems 110.

### B-2. Second Modification

The above embodiment describes the configuration that gives approval or disapproval for the use of medical information in the unit of each medical institution 100. Approval or disapproval may, however, be allowed to be given for each of the members (doctors and staff members) belonging to one medical institution 100. For example, when obtaining a selection instruction for selecting the first medical institution 100A as a subject medical institution 100 for approval or disapproval, the control unit 620 of the patient terminal device 600 causes a user interface shown in FIG. 9 to be displayed on the display unit 660. The user interface shown in FIG. 9 includes information on the members of the selected first medical institution 100A or more specifically information on teams provided in the first medical institution 100A and members of the respective teams. Checkboxes are provided near to individual team names and individual member names. When the patient desires to give approval for the entire first medical institution 100A, the patient selects an “OK” button without checking any of the checkboxes. An approval instruction indicating approval for the entire first medical institution 100A is accordingly sent to the information management device 400. In this case, the medical information of the patient is sent to the first information server 180A provided in the first information system 110A operated by the first medical institution 100A, like the embodiment described above.

When the patient does not desire to give approval for part of the respective teams or the respective members belonging to the first medical institution 100A, i.e., when the patient desires to give approval for only some part of the respective teams or the respective members belonging to the first medical institution 100A, on the other hand, the patient checks the checkbox corresponding to any team or any member to be excluded from approval and subsequently selects the “OK” button. An approval instruction indicating approval for the first medical institution 100A with suspension of approval for the checked team or members (hereinafter referred to as “excluded members or the like”) is sent to the information management device 400. This means that the patient expresses a disapproval intention to the excluded members or the like. In this case, the medical information of the patient is also sent to the first information server 180A provided in the first information system 110A operated by the first medical institution 100A. The first information system 110A, however, sets approval or disapproval for each team or each member to use the medical information of the patient and limits the use of the medical information of the patient by the excluded members or the like.

When the patient checks the checkboxes corresponding to all the teams or all the members belonging to the first information server 180A and selects the “OK” button in the user interface shown in FIG. 9, the control unit 420 of the information management device 400 may determine that disapproval for the first information server 180A is received. When the patient checks at least one checkbox corresponding to at least one team or member belonging to the first information server 180A and selects the “OK” button in the user interface shown in FIG. 9, the control unit 420 of the information management device 400 may determine that approval for the first information server 180A is received.

In the case where the medical information of the patient is stored in the first information server 180A, when the patient checks the checkboxes corresponding to part of the teams or members and selects the “OK” button in the user interface shown in FIG. 9, the control unit 420 of the information management device 400 notifies the first information system 110A of information for identifying the excluded members or the like without removing the medical information stored in the first information server 180A. In this case, the first information system 110A limits the use of the medical information of the patient by the excluded members or the like that are notified.

### B-3. Third Modification

The above embodiment describes the configuration that gives approval or disapproval for the use of medical information in the unit of each medical institution 100. Approval or disapproval may, however, be allowed to be given in the unit of a medical team constituting a plurality of medical institutions 100. The medical team is, for example, a collection of a hospital, medical clinics, dental clinics, dispensing pharmacies, and nursing facilities constituting a regional medical network and uses a common information system 110.

For example, when obtaining a selection instruction for selecting “team X” as a subject medical team for approval or disapproval, the control unit 620 of the patient terminal device 600 causes a user interface shown in FIG. 10 to be displayed on the display unit 660. The user interface shown in FIG. 10 includes information on medical institutions 100 (first medical institution 100A and second medical institution 100B) constituting the selected medical team “team X” and their members. Checkboxes are provided near to individual medical institution names and individual member names. When the patient desires to give approval for the entire medical team “team X”, the patient selects an “OK” button without checking any of the checkboxes. An approval instruction indicating approval for the entire medical team “team X” is accordingly sent to the information management device 400. In this case, the medical information of the patient is sent to an information server 180 provided in the information system 110 commonly used by the respective medical institutions constituting the medical team “team X”. This allows the medical information to be used by the respective medical institutions 100.

When the patient does not desire to give approval for part of the respective medical institutions or the respective members belonging to the medical team “team X”, i.e., when the patient desires to give approval for only some part of the respective medical institutions or the respective members belonging to the medical team “team X”, on the other hand, the patient checks the checkbox corresponding to any medical institution or any member to be excluded from approval and subsequently selects the “OK” button. An approval instruction indicating approval for the medical team “team X” with suspension of approval for the checked medical institution or members (hereinafter referred to as “excluded medical institution” and “excluded members”) is sent to the information management device 400. This means that the patient expresses a disapproval intention to the excluded medical institution or the excluded members. In this case, the medical information of the patient is also sent to the information server 180 provided in the information system 110 commonly used by the respective medical institutions constituting the medical team “team X”. This allows the medical information to be used by the respective medical institutions 100. The information system 110, however, sets approval or disapproval for each medical institution or each member to use the medical information of the patient and limits the use of the medical information of the patient by the excluded medical institution or the excluded members.

When the patient checks the checkboxes corresponding to all the medical institutions or all the members belonging to the medical team “team X” and selects the “OK” button in the user interface shown in FIG. 10, the control unit 420 of the information management device 400 may determine that disapproval for the medical team “team X” is received. When the patient checks at least one checkbox corresponding to at least one medical institution or member belonging to the medical team “team X” and selects the “OK” button in the user interface shown in FIG. 10, the control unit 420 of the information management device 400 may determine that approval for the medical team “team X” is received.

In the case where the medical information of the patient is stored in the information server 180 operated by the medical team “team X”, when the patient checks the checkboxes corresponding to part of the medical institutions or members and selects the “OK” button in the user interface shown in FIG. 10, the control unit 420 of the information management device 400 notifies the information system 110 of information for identifying the excluded medical institution or the excluded members without removing the medical information stored in the information server 180. In this case, the information system 110 limits the use of the medical information of the patient by the excluded medical institution or the excluded members that are notified.

### B-4. Fourth Modification

According to the embodiment described above, the medical information of each patient is generated and used by the medical institution 100. The medical information generated by the medical institution 100 may be allowed to be used by an institution other than the medical institution 100 (third-party institution). The following describes a fourth modification that allows the medical information of each patient to be used by an institution other than the medical institution 100.

FIG. 11 is a diagram illustrating the configuration of an information management system 10X according to the fourth modification. The information management system 10X of the fourth modification differs from the information management system 10 of the embodiment shown in FIG. 1 by that the information management system 10X includes a research information system 210 operated by a research institution 200 and a business information system 310 operated by another business institution 300, in addition to an information system 110 operated by a medical institution 100 (hereinafter referred to as “medical information system” 110 for the purpose of discrimination from the other information systems). The research information system 210 operated by the research institution 200 and the business information system 310 operated by the other business institution 300, as well as the medical information system 110 operated by the medical institution 100 are connected with the other devices and systems via a network NW. FIG. 11 illustrates one research institution 200 and one other business institution 300, but the number of research institutions 200 and the number of other business institutions 300 included in the information management system 10X may be two or more.

The research institution 200 is an institution that uses medical information to perform, for example, development of treatments and preventions of diseases and development of medical drugs and may be, for example, a laboratory, think tank, a university or a pharmaceutical company. The research information system 210 operated by the research institution 200 is a computer system configured to allow respective researchers, respective staff members and the like belonging to the research institution 200 to access to the medical information, so as to support the works of the respective researchers, the respective staff members and the like. The other business institution 300 denotes an institution using medical information other than the medical institution 100 and the research institution 200 and is an institution that uses medical information to perform various works, for example, a finance or insurance institution, an employment institution or an advertising institution. The business information system 310 operated by the other business institution 300 is a computer system configured to allow respective staff members and the like belonging to the other business institution 300 to access to the medical information, so as to support the works of the respective staff members and the like.

The research information system 210 of the research institution 200 includes a plurality of terminal devices 270 used by the respective staff members and the like and one or a plurality of research information servers 280 connected with the respective terminal devices 270 via a research institution intranet, like the medical information system 110. The research information server 280 includes a control unit 220, a memory unit 230 and a communication unit 240, like an information server 180 of the medical information system 110 (hereinafter referred to as “medical information server” 180 for the purpose of discrimination from the other information servers). Similarly, the business information system 310 of the other business institution 300 includes a plurality of terminal devices 370 used by the respective staff members and the like and one or a plurality of business information servers 380 connected with the respective terminal devices 370 via a business institution intranet. The business information server 380 includes a control unit 320, a memory unit 330 and a communication unit 340.

According to this modification, the research information system 210 of the research institution 200 and the business information system 310 of the other business institution 300 differ in a point that the research information system 210 and the business information system 310 use medical information without generating the medical information, from the medical information system 110 of the medical institution 100 that generates and uses medical information. The research information system 210 of the research institution 200 and the business information system 310 of the other business institution 300 may generate and use personal information other than medical information. One certain institution may fall under the category of two or more of the medical institution 100, the research institution 200 and the other business institution 300, and one certain institution may operate two or more of the medical information system 110, the research information system 210 and the business information system 310. Each of the research information system 210 of the research institution 200 and the business information system 310 of the other business institution 300 is one example of the sixth information system, the seventh information system, and the subject information system. The medical information system 110 of the medical institution 100 is one example of the fifth information system, the eighth information system and the ninth information system. The medical information is one example of specific personal information.

FIG. 12 is a diagram showing the flow of a personal information management process in the information management system 10X according to the fourth modification. FIG. 12 shows respective processes performed in a situation where a patient gives approval for the research information system 210 of the research institution 200 and the business information system 310 of the other business institution 300 to use medical information that is generated by the medical information system 110 of the medical institution 100 and that is stored in the medical information server 180 of the medical information system 110.

The respective processing details of FIG. 12 have much in in common with the respective processing details of FIG. 8. The following thus mainly and simply describes the differences from the respective processing details of FIG. 8. The control unit 620 of the patient terminal device 600 causes a user interface for selecting a subject institution for approval or disapproval, to be displayed on the display unit 660, and obtains a selection instruction for selecting one or a plurality of institutions via the operation unit 650 (S910). It is here assumed that the research institution 200 and the other business institution 300 are selected. The control unit 620 of the patient terminal device 600 sends the obtained selection instruction to the information management device 400 (S920).

When receiving the selection instruction sent from the patient terminal device 600, the control unit 420 of the information management device 400 sends approval information indicating an approval status of the patient X1 with respect to the institution (research institution 200 and other business institution 300) selected by the selection instruction, to the patient terminal device 600 (S930). The control unit 620 of the patient terminal device 600 causes the approval information sent from the information management device 400 to be displayed on the display unit 660. The control unit 620 of the patient terminal device 600 causes a user interface for giving approval or disapproval for the information system (research information system 210 and business information system 310) operated by the institution selected at S910 (research institution 200 and other business institution 300), to be displayed on the display unit 660, and obtains an approval instruction or a disapproval instruction via the operation unit 650 (S940).

According to the fourth modification, “approval” includes two different types of approvals, i.e., full approval that is approval for the use of the entire medical information and partial approval that is approval for the use of part of the medical information or more specifically for the use of partial medical information excluding at least one piece of attribute information included in the medical information. In general, medical information of each patient includes attribute information indicating the attributes of the patient (for example, name, sex, date of birth, address), in addition to substantial information (for example, results of medical tests). The attribute information may be regarded as information used to identify the patient (partial identification or full identification). With respect to approval for the research information system 210 and the business information system 310 to use the patient's own medical information, the patient X1 is allowed to select either full approval that is approval for the use of the entire medical information including the substantial information and the attribute information or partial approval that is approval for the use of the partial medical information excluding at least one piece of the attribute information.

FIG. 13 is a diagram illustrating one example of a user interface according to the fourth modification. For example, the user interface shown in FIG. 13 includes checkboxes for selection of either full approval or partial approval as the type of approval and checkboxes for selection of any of attribute information to be excluded in the case of selection of the partial approval. When the patient X1 desires to give approval for the use of the entire medical information including all the attribute information, the patient X1 is required to select “full approval”. When the patient X1 desires to give approval for the substantial information included in the medical information but desires to suspend approval for part or all of the attribute information included in the medical information, on the other hand, the patient X1 is required to select “partial approval” and select the attribute information as the subject of suspension of approval. When the full approval is selected, the entire medical information including all the attribute information is handed over, so that the medical information is used by the research information system 210 and the business information system 310 as real name information that allows for identification of the patient X1 that is the subject of the medical information. When the partial approval is selected, on the other hand, the partial medical information excluding at least one piece of the attribute information is handed over, so that the medical information is used as anonymous information (or partial anonymous information) that does not allow for (or does not partly allow for) identification of the patient X1 that is the subject of the medical information. FIG. 13 illustrates the user interface for giving approval for the research institution 200. A similar user interface is used to give approval for the other business institution 300. A user interface used may be configured to allow for collectively give approval for a plurality of institutions. The control unit 620 of the patient terminal device 600 sends the obtained approval instruction to the information management device 400 (S950).

When receiving the approval instruction sent from the patient terminal device 600, the control unit 420 of the information management device 400 updates an approval information table, based on the received approval instruction (S960) and gives the medical information server 180 provided in the medical information system 110 of the medical institution 100 that stores the medical information as the subject of the approval instruction, an instruction to send the medical information as the subject of the approval instruction to the storage server 500 (S970). For example, when the medical information as the subject of the approval instruction is partial medical information, the control unit 420 gives an instruction to send the partial medical information to the storage server 500.

When receiving the sending instruction, the control unit 120 of the medical information server 180 sends the medical information of the patient X1 to the specified storage server 500 (S980). For example, when the medical information as the subject of the approval instruction is partial medical information, the control unit 120 extract specified partial medical information from the medical information stored in the medical information server 180 and sends the extracted partial medical information to the storage server 500. The control unit 520 of the storage server 500 stores the received medical information of the patient X1 into the memory unit 530 (S990), and sends the medical information of the patient X1 to the research information server 280 and the business information server 380 (S1000). The control unit 220 of the research information server 280 (or the control unit 320 of the business information server 380) stores the received medical information of the patient X1 into the memory unit 230 (or into the memory unit 330) (S1010) and sends a notification on completion of storage of the medical information of the patient X1 to the information management device 400 (S1020).

When receiving the notification on completion of storage, the control unit 420 of the information management device 400 sends a permission notification for the use of the medical information of the patient X1, to the research information server 280 and the business information server 380 (S1030). When receiving the permission notification, the control unit 220 of the research information server 280 (or the control unit 320 of the business information server 380) sends a response notification to the information management device 400 (S1040). When receiving the response notification, the control unit 420 of the information management device 400 sends a notification on completion of sending the medical information to the patient terminal device 600 of the patient X1 (S1050). After that, the research information system 210 of the research institution 200 and the business information system 310 of the other business institution 300 allow each of the researchers, the staff members and the like belonging to the research institution 200 and the other business institution 300 to access and use the medical information of the patient X1 handed over from the medical information system 110 (S1060). For example, this enables the research institution 200 to perform, for example, development of treatments and preventions of diseases and development of medical drugs by using the medical information. This also enables the other business institution 300 to make various new business ventures and make innovation by using the medical information.

As described above, according to the fourth modification, the information system (medical information system 110) that generates and uses medical information and the information systems (research information system 210 and business information system 310) that use the medical information without generating the medical information are included as a plurality of information systems constituting the information management system 10X. When receiving approval of the patient X1 for the research information system 210 or the business information system 310 to use the medical information of the patient X1 that is generated by the medical information system 110, the control unit 420 of the information management device 400 causes the medical information of the patient X1 stored in the medical information server 180 provided in the medical information system 110 to be sent from the medical information server 180 to the storage server 500 and to be stored into the storage server 500. The control unit 420 of the information management device 400 then causes the medical information of the patient X1 stored in the storage server 500 to be sent from the storage server 500 to the research information server 280 or to the business information server 380 and to be stored into the research information server 280 or into the business information server 380 (fourth sending process). This configuration of the fourth modification allows the medical information that is generated by the information system (medical information system 110) generating and using medical information to be handed over to and used by the information systems (research information system 210 and business information system 310) using the medical information without generating the medical information. This configuration achieves the effective use of the medical information with approval of the patient.

According to the fourth modification, the approval for the use of medical information is either the full approval that is approval for the use of the entire medical information including all the attribute information or the partial approval that is approval for the use of the partial medical information excluding at least one piece of the attribute information from the medical information. When receiving the partial approval for the research information system 210 or the business information system 310 to use the partial medical information of the patient X1, the control unit 420 of the information management device 400 causes the partial medical information out of the medical information of the patient X1 that is stored in the medical information server 180 provided in the medical information system 110 to be sent from the medical information server 180 to the storage server 500 and to be stored into the storage server 500. The control unit 420 of the information management device 400 then causes the partial medical information of the patient X1 stored in the storage server 500 to be sent from the storage server 500 to the research information server 280 or to the business information server 380 and to be stored into the research information server 280 or into the business information server 380 (fifth sending process). This configuration of the fourth modification provides the option of approval for the use of the partial medical information excluding at least one piece of the attribute information from the medical information, in addition to the option of approval for the use of the entire medical information, as the possible options of approval for the use of the medical information. This configuration accelerates the effective use of the medical information, while protecting the privacy of the patient.

According to the fourth embodiment, in one receiving process, when receiving approval of the patient X1 for two or more subject information systems other than the medical information system 110 (for example, research information system 210 and business information system 310) to use the medical information of the patient X1 that is stored in the medical information server 180 provided in the medical information system 110, the control unit 420 of the information management device 400 causes the medical information of the patient X1 stored in the medical information server 180 to be sent from the medical information server 180 to the storage server 500 and to be stored into the storage server 500. The control unit 420 of the information management device 400 then causes the medical information of the patient X1 stored in the storage server 500 to be sent from the storage server 500 to the information servers (for example, research information server 280 and business information server 380) respectively provided in the above two or more subject information systems and to be stored into the respective information servers (sixth sending process). This configuration of the fourth modification receives the approval for the two or more subject information systems by one receiving process and allows the two or more subject information systems to use the medical information. This configuration enables the approval process and the transfer process of medical information to be performed efficiently. In general, the patient is more likely to select one (or a few) medical institution(s) and give approval for the use of medical information. The patient is, however, likely to select a large number of research institutions and the like and give approval for the use of medical information for the purpose of development of research or the like. The configuration of the fourth modification allows for efficient processing with respect to approval for a large number of institutions (information systems). This is especially effective in such cases.

### B-5. Other Modifications

According to the above embodiment, when receiving a sending request of location information issued by the patient terminal device 600, the control unit 420 of the information management device 400 generates location information showing a server where the medical information of the patient X1 is stored and sends the generated location information to the patient terminal device 600. A modification may cause the location information to be generated and sent to the patient terminal device 600 without receiving a sending request of the location information. For example, the control unit 420 of the information management device 400 may generate location information and send the generated location information to the patient terminal device 600 at regular intervals or at irregular intervals.

The above embodiment describes the process of, for example, sending and removing medical information of each patient, in response to receiving approval or disapproval of each patient for each information system 110 operated by each medical institution 100 to use the medical information of the patient. The present disclosure is similarly applicable to a process of, for example, sending and removing personal information of each user, in response to receiving approval or disapproval of each user for a general information system to use the personal information of the user. The information system may be, for example, an information system operated by a welfare facility, an information system operated by a healthcare institution or an information system operated by a net shop or a real shop. The personal information may be, for example, welfare information indicating, for example, the use status of welfare services, healthcare information indicating, for example, the results of medical checks, or purchase information indicating, for example, purchase history in a net shop or in a real shop.

The details of the personal information management process described above may be modified according to the type of the personal information. For example, information for identifying a protection level set for each type of personal information may be stored in the memory unit 430 of the information management device 400. With respect to personal information of a relatively high protection level (for example, medical information), a permission of an administrator of an information system that stores the personal information may be required, prior to transmission and removal of the personal information, under control of the control unit 420 of the information management device 400. The personal information may be allowed to be sent and removed only when the permission is obtained. With respect to personal information of a relatively low protection level (for example, purchase information), on the other hand, such a permission may not be required. This differs the requirement or non-requirement for the permission of transmission and removal of personal information according to the type of the personal information. This configuration improves the convenience and the flexibility in handling the personal information.

In a configuration that information correlating each information system to the type of personal information is stored in the memory unit 430 of the information management device 400 and that personal information is sent under control of the control unit 420 of the information management device 400, only the type of personal information correlated to an information system as a sending destination may be sent selectively. This configuration improves the convenience in handling the personal information.

In a configuration that information correlating the type of each information system (i.e., the type of an institution or a facility operating the information system) to the type of personal information is stored in the memory unit 430 of the information management device 400 and that personal information is sent under control of the control unit 420 of the information management device 400, only the type of personal information correlated to the type of an information system as a sending destination may be sent selectively. This configuration does not require to individually store the correlation of the individual information systems to the types of personal information and further improves the convenience in handling the personal information.

In a configuration that information correlating each storage server 500 to the type of personal information is stored in the memory unit 430 of the information management device 400 and that personal information is sent to the storage server 500 under control of the control unit 420 of the information management device 400, a storage server 500 correlated to the type of personal information to be sent may be selected, and the personal information may be sent to the selected storage server 500. This configuration enables various types of personal information to be stored in a plurality of storage servers 500 dispersedly. This reduces the volume of personal information stored in each of the storage servers 500 and achieves load distribution and reduction of the leakage risk.

According to the above embodiment, each of the information systems 110 is operated individually by one medical institution 100. One information system 110 may, however, be operated by a plurality of medical institutions 100 cooperate with each other.

According to the above embodiment, the control unit 420 of the information management device 400 receives approval or disapproval for the use of medical information of each patient via the network NW. The control unit 420 of the information management device 400 may, however, receive approval or disapproval via the operation unit 450.

Part of steps may be modified, may be omitted or may be exchanged with other steps in sequence in the personal information management process of the above embodiment. For example, a modification of the personal information management process shown in FIG. 2 may obtain and send an approval (or a disapproval) instruction (S140 and S150) without obtaining and sending a selection instruction for selecting a medical institution (S110 and 5120) and without sending approval information (S130). In this modification, the information of the approval information table AT stored in the information management device 400 is not sent to the patient terminal device 600. This modification may update the approval information table AT (S160) on the assumption that the approval instruction sent from the patient terminal device 600 to the information management device 400 has the right content. A modification of the personal information management process shown in FIG. 4 may allow medical information to be used (S650) when the medical information is stored into the second information server 180B without transmission of a permission notification from the information management device 400 to the second information server 180B (S620). These modifications are similarly applicable to the other flows of the personal information management process shown in the other drawings.

Although the present invention has been disclosed in the form of preferred embodiments and variations thereon, it will be understood that numerous additional modifications and variations could be made thereto without departing from the scope of the invention.

For the sake of clarity, it is to be understood that the use of “a” or “an” throughout this application does not exclude a plurality, and “comprising” does not exclude other steps or elements. The mention of a “unit” or a “module” does not preclude the use of more than one unit or module.

### REFERENCE SIGNS LIST

10: information management system, 100: medical institution, 110: medical information system, 120: control unit, 130: memory unit, 140: communication unit, 170: terminal device, 180: medical information server, 200: research institution, 210: research information system, 220: control unit, 230: memory unit, 240: communication unit, 270: terminal device, 280: research information server, 300: other business institution, 310: business information system, 320: control unit, 330: memory unit, 340: communication unit, 370: terminal device, 380: business information server, 400: information management device, 420: control unit, 430: memory unit, 440: communication unit, 450: operation unit, 460: display unit, 500: storage server, 520: control unit, 530: memory unit, 540: communication unit, 600: patient terminal device, 620: control unit, 630: memory unit, 640: communication unit, 650: operation unit, 660: display unit

1. An information management device connectable via a network with a storage server and with information servers respectively provided in a plurality of information systems, the information management device comprising:
a communication unit configured to make communication with an external device; and
a control unit, wherein
the control unit is configured to:
perform a receiving process of receiving approval or disapproval of each user for each of the information systems to use personal information of the user via the communication unit;
when receiving disapproval of a first user for a first information system that is one of the plurality of information systems, perform a first sending process of causing the personal information of the first user stored in a first information server provided in the first information system to be sent from the first information server to the storage server and to be stored into the storage server; and
when receiving approval of the first user for a second information system that is one of the plurality of information systems, perform a second sending process of causing the personal information of the first user stored in the storage server to be sent from the storage server to a second information server provided in the second information system and to be stored into the second information server.

2. The information management device according to claim 1,
wherein the control unit is configured to, when receiving the disapproval of the first user for the first information system, perform a first removal process of removing the personal information of the first user from the first information server.

3. The information management device according to claim 1,
wherein the control unit is configured to, when receiving the approval of the first user for the second information system, perform a second removal process of removing the personal information of the first user from the storage server.

4. The information management device according to claim 1,
wherein the control unit is configured to, when receiving approval of a second user for a third information system that is one of the plurality of information systems, performing the second sending process of causing the personal information of the second user to be stored into a third information server provided in the third information system, and subsequently receiving approval of the second user for a fourth information system that is one of the plurality of information systems, perform a third sending process of causing the personal information of the second user stored in the third information server to be sent from the third information server to the storage server, to be further sent from the storage server to a fourth information server provided in the fourth information system, and to be stored into the fourth information server.

5. The information management device according to claim 1,
the control unit receives the approval or the disapproval via the network in the receiving process.

6. The information management device according to claim 1,
wherein on satisfaction of a predetermined condition, the control unit sends location information for identifying the information server and the storage server where the personal information of a third user is stored, via the communication unit to a terminal device of the third user that is the external device.

7. The information management device according to claim 6,
wherein the control unit is configured to perform a request receiving process of receiving a sending request of the location information from the terminal device of the third user via the communication unit, wherein
the predetermined condition is that the sending request of the location information is received.

8. The information management device according to claim 1,
wherein the control unit is configured to:
when receiving the disapproval of the first user for the first information system, perform a determination process of determining whether type of the personal information of the first user is a first type or a second type; and
when determining that the type of the personal information of the first user is the second type, perform a request process of requesting the first information system to give a permission for sending the personal information of the first user from the first information server, wherein
the control unit performs the first sending process on a condition that the permission is received from the first information system.

9. The information management device according to claim 1, further comprising:
a memory unit configured to store a first table that is configured to specify a correlation of each of the plurality of information systems to each of multiple different types of the personal information, wherein
the control unit is configured to, when receiving the approval of the first user for the second information system, perform an identification process of identifying the type of the personal information as a subject of the approval, wherein
the control unit performs the first sending process on a condition that the identified type of the personal information is correlated to the second information system by the first table.

10. The information management device according to claim 9,
wherein the first table is configured to specify the correlation of each of the plurality of information systems to each of the multiple different types of the personal information by specifying a correlation of each of multiple different types of the information systems to each of the multiple different types of the personal information.

11. The information management device according to claim 1, further comprising:
a memory unit configured to store a second table that is configured to specify a correlation of each of a plurality of the storage servers to each of a plurality of the users, wherein
the control unit sends the personal information of the first user to the storage server correlated to the first user by the second table in the first sending process.

12. The information management device according to claim 1, further comprising:
a memory unit configured to store a third table that is configured to specify a correlation of each of a plurality of the storage servers to each of multiple different types of the personal information, wherein
the control unit sends the personal information of the first user to the storage server correlated to the type of the personal information of the first user by the third table in the first sending process.

13. The information management device according to claim 1,
wherein the personal information includes medical information.

14. The information management device according to claim 1,
wherein each of the information systems allows a plurality of members to use the information server, and
the control unit receives approval or disapproval for each of the members in the receiving process,
when receiving the disapproval for all the members allowed to use the first information server, the control unit determines that the disapproval for the first information system is received, and
when receiving the approval for at least one of the members allowed to use the second information server, the control unit determines that the approval for the second information system is received.

15. The information management device according to claim 1,
wherein the plurality of information systems includes:
a fifth information system configured to generate and use specific personal information that is a specific type of the personal information, and
a sixth information system configured to use the specific personal information without generating the specific personal information, wherein
the control unit is configured to, when receiving approval of a fourth user for the sixth information system to use the specific personal information of the fourth user generated by the fifth information system, perform a fourth sending process of causing the specific personal information of the fourth user stored in a fifth information server provided in the fifth information system to be sent from the fifth information server to the storage server and to be stored into the storage server, and causing the specific personal information of the fourth user stored in the storage server to be sent from the storage server to a sixth information server provided in the sixth information system and to be stored into the sixth information server.

16. The information management device according to claim 1,
wherein the personal information includes at least one piece of attribute information indicating an attribute of each user, and
the approval received in the receiving process is either full approval that is approval for use of the personal information including all the attribute information or partial approval that is approval for use of partial personal information excluding at least one piece of the attribute information from the personal information, wherein
the control unit is configured to, when receiving partial approval of a fifth user for a seventh information system that is one of the plurality of information systems to use the partial personal information of the fifth user, perform a fifth sending process of causing the partial personal information out of the personal information of the fifth user stored in an eighth information server provided in an eight information system that is one of the plurality of information systems to be sent from the eighth information server to the storage server and to be stored into the storage server, and causing the partial personal information of the fifth user stored in the storage server to be sent from the storage server to a seventh information server provided in the seventh information system and to be stored into the seventh information server.

17. The information management device according to claim 1,
wherein the control unit is configured to, when receiving approval of a sixth user for two or more subject information systems that are included in the plurality of information systems and are other than a ninth information system that is one of the plurality of information systems, to use the personal information of the sixth user that is stored in a ninth information server provided in the ninth information system in one cycle of the receiving process, perform a sixth sending process of causing the personal information of the sixth user stored in the ninth information server to be sent from the ninth information server to the storage server and to be stored into the storage server, and causing the personal information of the sixth user stored in the storage server to be sent from the storage server to information servers respectively provided in the two or more subject information systems and to be stored into the information servers.

18. An information management system, comprising:
a storage server;
information servers respectively provided in a plurality of information systems; and
the information management device according to claim 1 wherein each of the information servers comprises:
a system communication unit and
a system control unit, wherein
the control unit of the information management device receives approval or disapproval for each member in the receiving process and sends approval information indicating either the approval or the disapproval to a corresponding information system, and
the system control unit of the information system is configured to:
perform an information receiving process of receiving the approval information via the system communication unit and
when receiving the approval information, perform a setting process of setting approval or disapproval for each member to use the personal information of the user.

19. (canceled)

20. An information management method, comprising:
a process of receiving approval or disapproval of each user for each of a plurality of information systems to use personal information of the user via a network;
in response to receiving disapproval of a first user for a first information system that is one of the plurality of information systems, a process of causing the personal information of the first user stored in a first information server provided in the first information system to be sent from the first information server to a storage server and to be stored into the storage server; and
in response to receiving approval of the first user for a second information system that is one of the plurality of information systems, a process of causing the personal information of the first user stored in the storage server to be sent from the storage server to a second information server provided in the second information system and to be stored into the second information server.

21. A computer program configured to cause an information management device to perform:
a process of receiving approval or disapproval of each user for each of a plurality of information systems to use personal information of the user via a network;
in response to receiving disapproval of a first user for a first information system that is one of the plurality of information systems, a process of causing the personal information of the first user stored in a first information server provided in the first information system to be sent from the first information server to a storage server and to be stored into the storage server; and
in response to receiving approval of the first user for a second information system that is one of the plurality of information systems, a process of causing the personal information of the first user stored in the storage server to be sent from the storage server to a second information server provided in the second information system and to be stored into the second information server.
